(19) United States
(12) Patent Application Publication
(10) Pub. No.: US 2015/0150442 A1
(43) Pub. Date: Jun. 4, 2015

(54) ENDOVASCULAR PROBE

(71) Applicant: The Regents of the University of California, Oakland, CA (US)

(72) Inventors: Bashir A. Tafti, Los Angeles, CA (US); Edward W. Lee, Los Angeles, CA (US); Stephen T. Kee, Santa Monica, CA (US)

(21) Appl. No.: 14/405,767

(22) PCT Filed: Jun. 4, 2013
(86) PCT No.: PCT/US2013/044136

§ 371 Date: Dec. 4, 2014

Related U.S. Application Data
(60) Provisional application No. 61/655,783, filed on Jun. 5, 2012.

(51) Int. Cl. A61B 1/313; A61B 18/24; A61B 17/12; A61B 1/018; A61B 1/05; A61B 1/00; A61B 1/015; A61M 3/02; A61M 25/00

(52) U.S. Cl. CPC A61B 1/3137; A61M 3/027; A61B 18/245; A61B 17/12136; A61M 25/0074; A61M 25/0026; A61B 1/05; A61B 1/00082; A61B 1/00117; A61B 1/015; A61B 1/00094; A61B 1/018; A61B 2018/00982

(57) ABSTRACT

An embodiment of an endovascular probe includes a probe body, an inflatable mechanism surrounding a portion of the probe body and configured to create a reversible occlusion within a vascular system, multiple channels extending through the probe body, and an imaging device positioned within one of the channels. Another embodiment of an endovascular probe includes a probe body with a first channel configured to accommodate an instrument, a second channel configured to accommodate an imaging device, and a third channel configured to accommodate an irrigation device, along with an inflatable device at an external periphery of the probe body. Another embodiment of an endovascular probe includes an image recording device, a bi-modal mechanism that selectively provides one of irrigation and suction, and a laser fiber.

### CROSS-REFERENCE TO RELATED APPLICATION

This application claims the benefit of U.S. Provisional Patent Application 61/655,783 filed Jun. 5, 2012 to Tafti et al., entitled “Endovascular Lithotripsy,” which is incorporated by reference herein in its entirety.

### BACKGROUND

Use of an endovascular probe in a vascular environment presents many challenges, such as maneuvering and operating in continuous blood flow within small diameter vessels. The endovascular probe described in this disclosure overcomes challenges of the vascular environment by providing for flexible positioning, image feedback, irrigation and suction, and controllable occlusion, among other benefits.

### SUMMARY

An embodiment of an endovascular probe includes a probe body, an inflatable mechanism surrounding a portion of the probe body and configured to create a reversible occlusion within a vascular system, multiple channels extending through the probe body, and an imaging device positioned within one of the channels. The imaging device may detect a pattern of energy. The imaging device may include a charge-coupled device. The probe may further include an illumination source positioned within one of the channels, and information representing an image detected by the imaging device may be provided externally to the probe body by transmitting the information through a fiber, and energy for the illumination source may be received through the fiber. The probe may further include an irrigation device positioned within one of the channels, and the irrigation device may be selectively configured to provide irrigation or suction. The probe may further include a suction device positioned within one of the channels. The probe body may have a tip that is selectively movable. The probe may further include a laser fiber positioned within one of the channels, wherein the laser fiber is selectively movable. A maximum diameter of the probe body may be less than two centimeters. The probe may further include an extendable mechanism positioned within one of the channels and configured to selectively extend from the probe body, the extendable mechanism including an inflatable component at a distal end of the extendable mechanism.

Another embodiment of an endovascular probe includes a probe body with a first channel configured to accommodate an instrument, a second channel configured to accommodate an imaging device, and a third channel configured to accommodate an irrigation device, along with an inflatable device at an external periphery of the probe body. The instrument may be a laser. The inflatable device may be a first inflatable device, and the instrument may include a second inflatable device positioned at a distal end of an extendable mechanism. The irrigation device may selectively provide irrigation or suction. The imaging device may be a charge-coupled device semiconductor chip. The probe may further include a fourth channel configured to accommodate a guidewire.

Another embodiment of an endovascular probe includes an imaging device, a bi-modal mechanism that selectively provides one of irrigation and suction, and a laser fiber. The probe may further include an extendable inflation device. The probe may further include a body member surrounding the imaging device, bi-modal mechanism, and laser fiber, and a balloon connected to the body member and configured to hold the body member in a fixed endovascular position when inflated.

A method of using an endovascular probe includes receiving images of an interior of a vessel from an imaging device positioned within the probe, irrigating the vessel, suctioning the vessel, maneuvering the probe to a target position within the vessel based on the received images, and providing an inflation medium to an inflatable device positioned on the exterior of the probe until the inflatable device is sufficiently inflated to maintain the position of the probe within the vessel. Receiving the images may include detecting a pattern of energy, and may further include recording the pattern of energy. The imaging device may include a charge-coupled device. The method may further include illuminating an area proximate the target position by an illumination source positioned within the probe. The method may further include providing information representing an image detected by the imaging device by transmitting the information through a fiber, and may also include receiving energy for an illumination source through the fiber. The method may further include delivering an irrigation fluid from an irrigation device positioned within the probe. The irrigation device may be selectively configured to provide irrigation or suction. The method may further include suctioning using a suction device positioned within the probe. The method may further including selectively moving a tip of the probe, and positioning the tip for improved placement of an instrument. The method may further include activating a laser through a laser fiber positioned within the probe. The method may further include controlling a position of a laser fiber within a channel of the probe. Maneuvering may include maneuvering the probe through a vessel with a diameter less than two centimeters, or through a sheath for which a maximum outer diameter is less than two centimeters. The method may further include extending an extendable mechanism from within a channel of the probe. The extendable mechanism may include an inflatable component at a distal end of the extendable mechanism.

Another method of using an endovascular probe includes providing a probe body with a first channel configured to accommodate an instrument, a second channel configured to accommodate an imaging device, and a third channel configured to accommodate an irrigation device, the method also including attaching a first inflatable device at an external periphery of the probe body. The method may include positioning an instrument within the first channel. The instrument may be a laser. The instrument may include a second inflatable device positioned at a distal end of an extendable mechanism, and the method may include controllably extending the extendable mechanism from the probe body. The method may include positioning an irrigation device within the third channel. The method may further include controlling the irrigation device to selectively provide irrigation or suction. The imaging device may be a charge-coupled device semiconductor chip. The method may include providing a fourth channel in the probe body configured to accommodate a guidewire.

Another method of using an endovascular probe includes providing an imaging device, a bi-modal mechanism that selectively provides one of irrigation and suction, and a laser fiber, and the method further includes controlling the bi-modal mechanism to remove fluid and particles from an area proximate the probe and thereby provide an improved field of view within a vessel. The method further includes receiving images of the field of view from the imaging device. The probe may include a body member surrounding the imaging device, bi-modal mechanism, and laser fiber, and may further include a balloon connected to the body member. The method may include providing an inflation medium to the balloon, thereby firmly positioning the probe within the vessel. The method may further include controlling an extendable inflation device to extend from the probe.

### BRIEF DESCRIPTION OF THE DRAWINGS

FIG. 1A-FIG. 1C illustrate various views of an example of an embodiment of an endovascular probe.

FIG. 1D illustrates an example of an embodiment of an endovascular probe.

FIG. 1E illustrates an example of an embodiment of an endovascular probe.

FIG. 2 illustrates an example of using an embodiment of an endovascular probe in a procedure.

FIG. 3 illustrates an example of an embodiment of an endovascular probe.

FIG. 4A illustrates an example of using an embodiment of an endovascular probe in a procedure.

FIG. 4B illustrates an example of using an embodiment of an endovascular probe in a procedure.

### DETAILED DESCRIPTION

The endovascular probe described in this disclosure provides for flexible positioning, image feedback, irrigation and suction, and controllable occlusion, among other benefits.

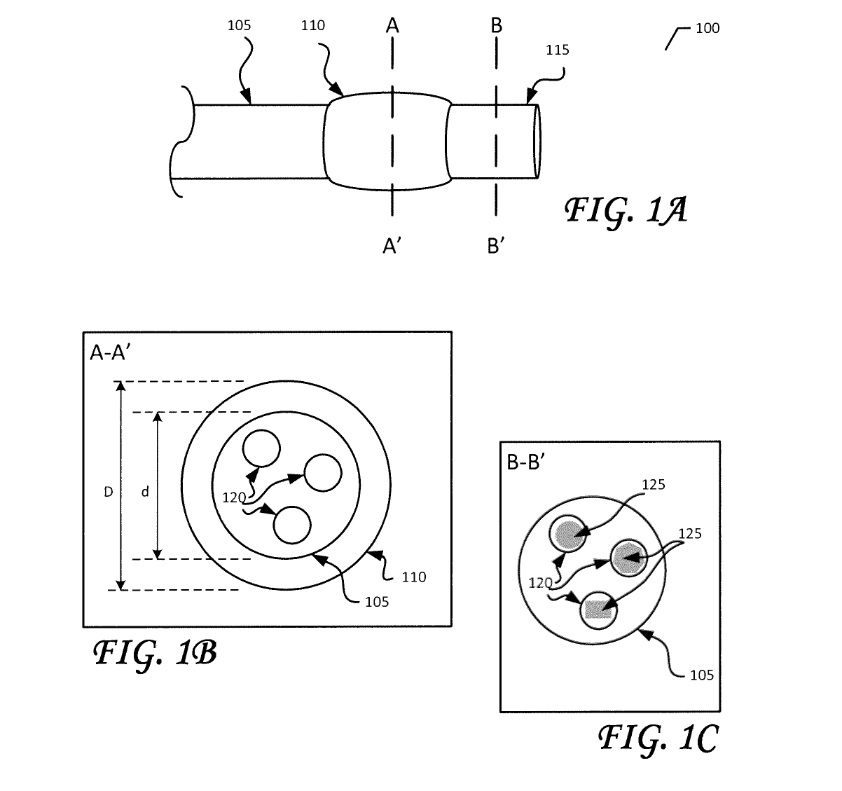

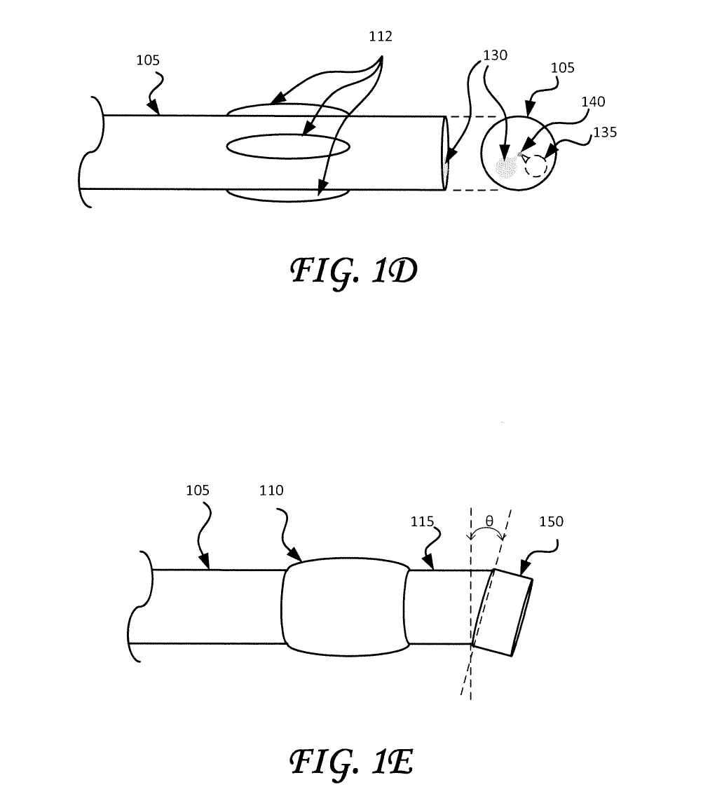

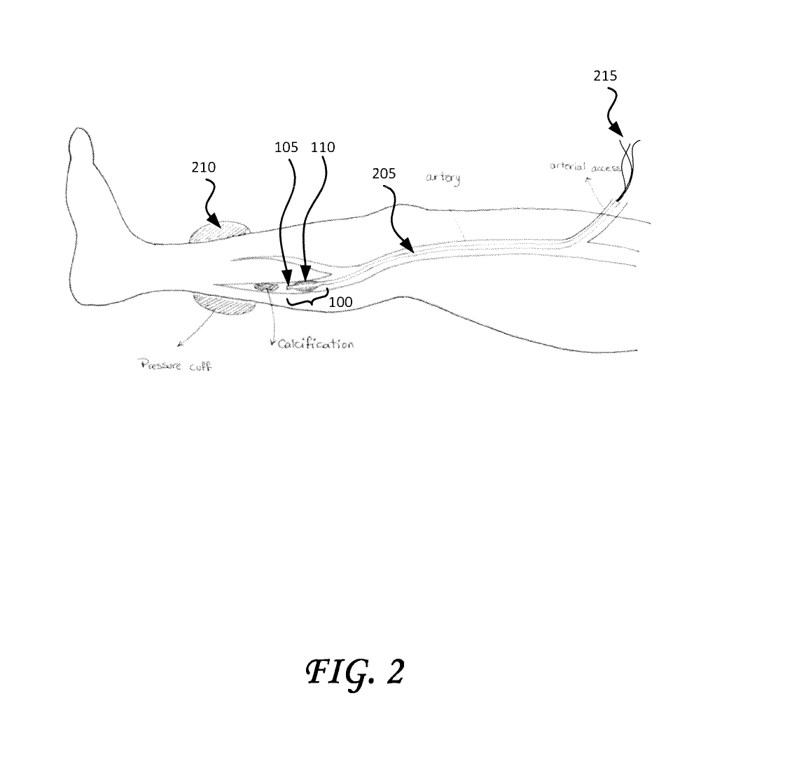

FIG. 1A-FIG. 1C illustrate an example of an endovascular probe 100 according to one embodiment of this disclosure. Probe 100 includes a generally cylindrical probe body 105, and an inflatable mechanism 110 attached to probe body 105 at an external periphery of probe body 105. Referring to FIG. 1B showing a cross sectional view along A-A′ of FIG. 1A, inflatable mechanism 110 may extend around the circumference of probe body 105. In other implementations, inflatable mechanism 110 may extend around a portion of the circumference of probe body 105. In its inflated state, a diameter D (or other cross-sectional dimension) of inflatable mechanism 110 may be at least 1.1 times a diameter d (or other cross-sectional dimension) of probe body 105, such as at least about 1.2 times, at least about 1.3 times, at least about 1.4 times, or at least about 1.5 times diameter d.

Also shown in cross-sectional view A-A′ of FIG. 1B are channels 120 in probe body 105. Channel 120 may extend partially or fully through the length of probe body 105. An example of a channel 120 extending partially through probe body 105 is a cavity containing an imaging device, where a wireless interface transmits image information from the imaging device to an external receiver. Three channels are shown in FIG. 1B by way of illustration. However, additional or fewer channels may be included. The positioning of channels 120 within probe body 105 may be as illustrated in FIG. 1B, but may be modified for various implementations.

Referring again to FIG. 1A, along with a cross-section view along B-B′ of FIG. 1A as shown in FIG. 1C, a portion 115 of probe body 105 is not in contact with and extends beyond inflatable device 110. The length of portion 115 may be, for example, about 5 centimeters (cm) or less, about 4 cm or less, about 3 cm or less, or about 1 cm or less, and may be down to, for example, about 0.5 cm, about 0.2 cm, or about 0.1 cm. The length of portion 115 may be within a range, such as between 1.5 cm and 3 cm. In some implementations, inflatable device 110 extends the length of probe body 105.

FIG. 1C further illustrates that devices 125 may be positioned within channels 120. Devices 125 may include, for example, an illumination source, an imaging device, a viewing device, an irrigation device, a suction device, a laser, or other instrument. Device 125 may be one of many possible shapes and sizes, may fully or partially fill a diameter of channel 120, and may be recessed from, even with, or protruded from probe body 105. Device 125 may be inserted and locked into place within channel 120, or may remain movable within channel 120. Device 125 may be permanently affixed to channel 120. In some implementations, for an unused channel 120, device 125 may be a plug to prevent fluids from entering probe 100 through unused channel 120.

FIG. 1D illustrates that, in some implementations, inflatable mechanism 110 may be constructed of multiple inflatable portions 112 positioned around the circumference of probe body 105. As also illustrated in FIG. 1D, probe 100 may include a cover mechanism for selectively covering all or a portion of the tip of probe 100 during a procedure, such as for covering a scope lens during initial placement of probe 100 or during an irrigation process. In FIG. 1D, an example of a cover mechanism 130 is illustrated, that is controllably rotated from a position 135 about pivot point 140. Other cover mechanisms 130 include, for example, a shutter-type cover mechanism 130 over channel 120, a cover mechanism 130 that moves from within channel 120 to cover the opening of channel 120, or a cover mechanism 130 that is positioned over the opening of channel 120 that is pushed out of the way by deployment of a device 125.

FIG. 1E illustrates that probe 100 may include a selectively movable tip 150. For example, when probe 100 is positioned and inflatable device 110 is inflated, movable tip 150 may be rotated or tilted to achieve a desirable positioning of instruments in relation to the procedure area. Movable tip 150 is illustrated as moving through an angle θ in FIG. 1E. Movable tip 150 is further illustrated as having a diameter larger than the diameter of portion 115 such that the internal perimeter of movable tip 150 is outside of the external perimeter of portion 115. Instead, movable tip 150 may be configured such that movable tip 150 has a diameter smaller than the diameter of portion 115 such that the external perimeter of movable tip 150 is inside of the internal perimeter of portion 115. In one implementation, movable tip 150 is shaped spherically or hemispherically.

In another embodiment, the focal position or distance of the imaging mechanism may be adjusted. For example, a flexible or movable lens may be used.

Additionally or alternatively to movable tip 150, probe 100 may include one or more selectively movable instruments in channels 120. For example, levers may be positioned along channel 120 to move an instrument within channel 120, and a hinge mechanism may be included at the end of probe 100 to allow the instrument to move in one or more directions.

One implementation of probe 100 includes three channels 120, in which a first channel accommodates a camera, a second channel accommodates a combination irrigation/suction mechanism, and a third channel accommodates passage of instruments appropriate for the particular vascular procedure. One such instrument is a holmium laser fiber, which may be used, for example, for ablation, cauterization, or incising.

One implementation of probe 100 includes four channels 120, in which a first channel accommodates a camera, a second channel accommodates an irrigation mechanism, a third channel accommodates a suction mechanism, and a fourth channel accommodates passage of instruments appropriate for the particular vascular procedure. One or both of the irrigation and suction mechanisms may be selectively bi-modal, meaning selectively providing irrigation or suction, thereby providing the capability to remove material that may become lodged within the irrigation or suction mechanism, respectively. Thus, for example, the irrigation mechanism may be cycled between irrigation and suction to remove debris in the irrigation mechanism.

One implementation of probe 100 includes an additional channel for an illumination source (e.g., a light source) to illuminate a target vascular area. In an alternative configuration, an illumination source is provided on a fiber that also carries information from an imaging device in probe 100.

Another implementation of probe 100 includes an additional channel for a guidewire.

In another implementation, the body of probe 100 may be telescoping to extend the length of probe 100.

FIG. 2 illustrates an example of how probe 100 may be used in a vascular procedure such as in peripheral arterial lithotripsy. Probe 100 is shown with sheath 205, which may be a sheath 205 through which probe 100 is extended, a sheath 205 attached to probe 100, or a sheath 205 formed as part of probe 100. For example, sheath 205 may be maneuvered proximate a target position such as a vascular occlusion, and probe 100 subsequently deployed from sheath 205.

As illustrated in the example of FIG. 2, probe 100 is inserted through an arterial access point into an artery, to a target position. Probe 100 may be guided to the target position using an imaging device in channel 120. An imaging device may record patterns of energy as images, and transmit information representing the images for accurate maneuvering and positioning of probe 100, and for visualization of the vascular area after positioning. Information may be transferred optically, electrically, or wirelessly. An imaging device may include, for example, a charge-coupled device (CCD) semiconductor chip. Improved clarity of image may be achieved using an irrigation device and/or a suction device to remove blood and debris proximate the imaging device during and after positioning. Irrigation and/or suction may be continuous. Once probe 100 is positioned, inflatable mechanism 110 is inflated, mitigating against blood flow from a proximal arterial supply. Also illustrated in FIG. 2 is an optional pressure cuff 210 for mitigation against regurgitation of blood from a distal blood pool after an occlusion is removed, and for mitigation against embolization of residual debris to a distal arterial tree.

Probe 100 may include one or more conduits attached to a distal end of probe 100. For example, a conduit may be an optical fiber for transmitting light to probe 100 or for transmitting image information from probe 100, a channel for providing irrigation or suction to or from probe 100, respectively, or a channel for passing an instrument or a guidewire. In FIG. 2, conduits 215 are connected to probe 100 and extend through sheath 205. Conduits 215 may be coupled to external equipment, such as viewing devices, pumps, lasers, and the like.

In a vascular procedure for treating an occlusion, substantially continuous irrigation and suction through probe 100 acts to remove blood trapped between inflatable mechanism 110 and the occlusion. The blood may be suctioned with a suctioning device in probe 100 and replaced by a clear medium such as saline using an irrigation device in probe 100. The occlusion may be visualized by an imaging device in probe 100, and destroyed by a laser in probe 100. The resulting debris may be suctioned.

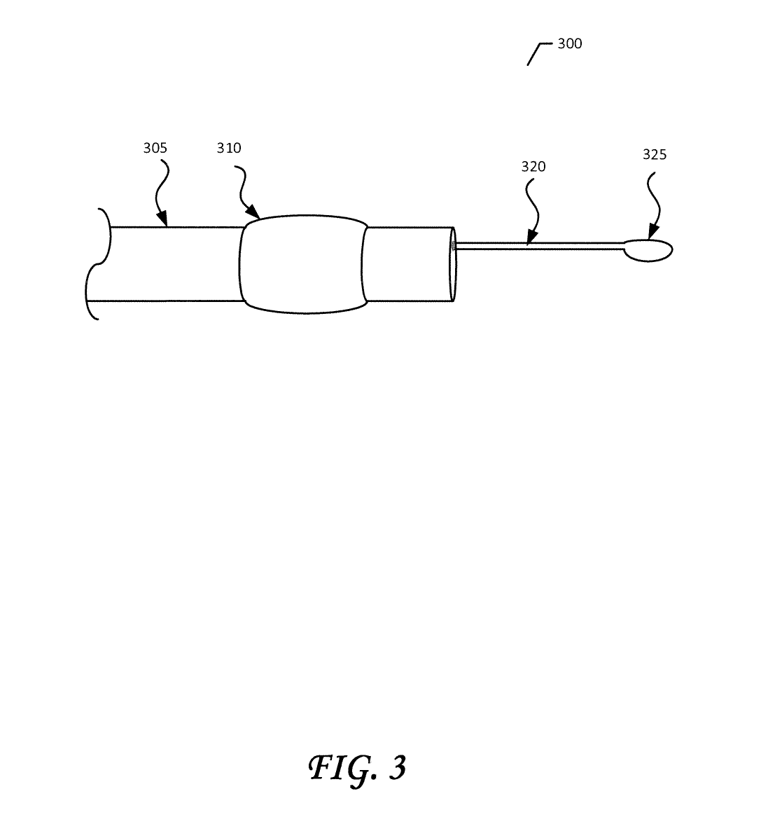

FIG. 3 illustrates an example of an endovascular probe 300, which is similar to probe 100 except that probe 300 includes an instrument which is a distal inflatable device 325 with distal arm 320 in a channel of probe body 305. Distal arm 320 may be extendable from within probe 300. Probe 300 further includes an inflatable device 310 around probe body 305 for creating a reversible occlusion.

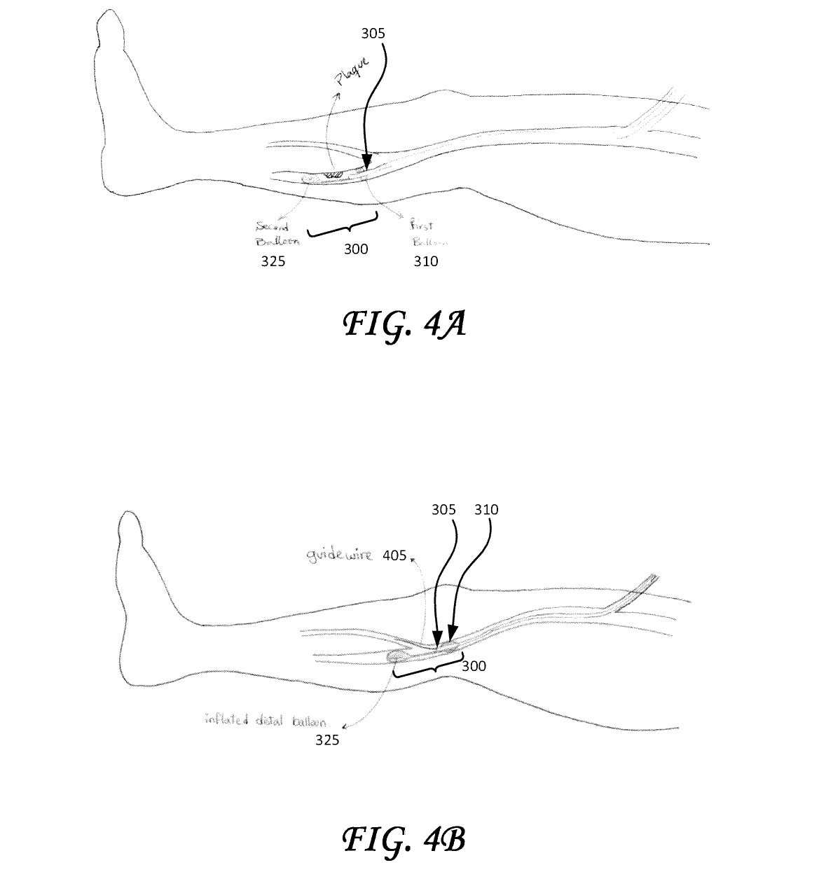

FIG. 4A illustrates an example of the use of probe 300 in a procedure. In this example, inflatable devices 310 and 325 are balloons. Probe 300 is inserted through an arterial access and positioned using imaging. Inflatable device 310 is inflated to occlude a proximal supply vessel, and distal inflatable device 325 is inflated to create a complete occlusion distal to a partially occluding lesion. Such positioning may be useful when the device is used for ablation of partially occluding plaques or when a section of the lumen becomes opened through ablation of a segment of a fully occluding lesion.

FIG. 4B illustrates an example of the use of probe 300 in another procedure. In this example, probe 300 includes a guidewire 405. Probe 300 is inserted through an arterial access and positioned at a junction of two vessels using guidewire 405. Inflatable device 310 is inflated to occlude a proximal supply vessel, and distal inflatable device 325 is inflated to mitigate against blood flow regurgitation from a blood pool.

In another implementation, inflatable device 325 is positioned on a telescoping segment such that the length of the body of probe 300 distal to inflatable device 325 can be changed, allowing for keeping the target in focus and providing for a larger field of operation without the need to deflate-inflate either of inflatable devices 310 or 325.

In another implementation, the body of probe 300 may be telescoping to extend the length of probe 100.

The endovascular probes described with respect to FIGS. 1-4 are provided by way of example and are not limiting. Other implementations are also within the scope of this disclosure. Generally, a probe may include one or more of an illumination source, an imaging device, a viewing device, an irrigation device, a suction device, a laser, and other instruments. Further, a probe may include multiple instances of one or more of a light source, an imaging device, a viewing device, an irrigation device, a suction device, a laser, and other instruments. In some implementations, a probe may be guided using a guidewire, and in other implementations, a probe may be guided using feedback from an imaging device instead of using a guidewire. In some implementations, both an imaging device and a guidewire may be used.

Correspondingly to the variety of possible implementations of a probe, the probe body may contain a variety of channels, which may be of different cross-sectional area, or may be of substantially the same cross-sectional area. Further, a probe may have a diameter of two cm or less to allow for use in vascular branches, such as about 1.8 cm or less, about 1.6 cm or less, or about 1.4 cm or less. Position of channels within the probe body may be optimized for a procedure. For example, a channel may be positioned near an outer edge of the probe body for placement of a light source, providing for better light distribution and better image quality. For another example, a 350 micron diameter channel may be included in the probe body for a laser fiber, and other larger diameter channels included for other instruments.

The length of an endovascular probe as described in this disclosure varies, dependent on, for example, intended use, convenience of use, limitations of instruments or associated equipment, and whether the probe includes a sheath. For example, probe length may be limited by the length of a laser fiber that is tuned for a particular wavelength. For another example, probe length may be limited by a bandwidth limitation of an optical fiber used in transmitting images from an imaging device. For a further example, probe length may be limited by the capability of an irrigation or suction pump. In some embodiments, an endovascular probe is between 80 cm and 1.5 meters in length, inclusive of instruments and related conduits, which may be incorporated as part of the probe body. In some embodiments, the probe body is substantially smaller than the assembled probe length, such 5 cm or less, 10 cm or less, or 15 cm or less.

Thus has been described minimally-invasive endovascular probes providing imaging capability for maneuvering and positioning the probe, and for viewing a procedure area. The imaging capability allows the probe to be maneuvered and positioned with or without a guidewire. An inflatable mechanism attached to the probe allows the probe to stay where positioned, and further creates a reversible occlusion for stopping blood flow, allowing for better viewing of the procedure area. Channels in the probe provide for instruments to be positioned within the probe or passed through the probe. Instruments include but are not limited to light sources, imaging devices, laser fibers, irrigation devices, suction devices, bi-modal suction/irrigation devices, and additional inflatable devices for creating reversible occlusions in vessels proximate the procedure area. A channel may provide for the passage of a guidewire.

Embodiments of this disclosure relate to methods and devices for vascular treatments. Examples of commercial products are probes to be used by Interventional Radiologists for ablation of peripheral vascular calcifications or other obstructions or occlusions.

Embodiments of this disclosure relate to the application of holmium laser lithotripsy.

While the invention has been described with reference to the specific embodiments thereof, it should be understood by those skilled in the art that various changes may be made and equivalents may be substituted without departing from the true spirit and scope of the invention as defined by the appended claims. In addition, many modifications may be made to adapt a particular situation, material, composition of matter, method, operation or operations, to the objective, spirit and scope of the invention. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations is not a limitation of the invention.

What is claimed is:

1. An endovascular probe, comprising;
a probe body;
an inflatable mechanism surrounding a portion of the probe body and configured to create a reversible occlusion within a vascular system;
a plurality of channels extending at least partially through the probe body; and
an imaging device configured to be positioned within one of the plurality of channels.

2. The endovascular probe in claim 1, wherein the imaging device is configured to detect a pattern of energy.

3. The endovascular probe in claim 2, wherein the imaging device includes a charge-coupled device.

4. The endovascular probe in claim 1, further comprising an illumination source configure to be positioned within another one of the plurality of channels.

5. The endovascular probe in claim 4, wherein information representing an image detected by the imaging device is provided externally to the probe body by transmitting the information through a fiber, and wherein energy for the illumination source is further received through the fiber.

6. The endovascular probe in claim 1, further comprising an irrigation device configured to be positioned within another one of the plurality of channels.

7. The endovascular probe in claim 6, wherein the irrigation device is selectively configured to provide one of irrigation and suction.

8. The endovascular probe in claim 6, further comprising a suction device positioned within another one of the plurality of channels.

9. The endovascular probe in claim 1, the probe body having a tip at a distal end, wherein the tip is selectively movable.

10. The endovascular probe in claim 1, further comprising a laser fiber positioned within another one of the plurality of channels, wherein the laser fiber is selectively movable.

11. The endovascular probe in claim 1, wherein a maximum diameter of the probe body is less than two centimeters.

12. The endovascular probe in claim 1, further comprising an extendable mechanism positioned within another one of the plurality of channels and configured to selectively extend from the probe body, the extendable mechanism including a distal arm and an inflatable component at a distal end of the distal arm.

13. An endovascular probe, comprising:
a probe body including a first channel configured to accommodate an instrument, a second channel configured to accommodate an imaging device, and a third channel configured to accommodate an irrigation device; and
an inflatable device at an external periphery of the probe body.

14. The endovascular probe in claim 13, further comprising an instrument, wherein the instrument is a laser fiber.

15. The endovascular probe in claim 13, further comprising an instrument, wherein the inflatable device is a first inflatable device, and wherein the instrument is an extendable mechanism including a second inflatable device positioned at a distal end of the extendable mechanism.

16. The endovascular probe in claim 13, further comprising an irrigation device, wherein the irrigation device selectively provides one of irrigation and suction.

17. The endovascular probe in claim 13, further comprising an imaging device, wherein the imaging device is a charge-coupled device semiconductor chip.

18. The endovascular probe in claim 13, further comprising a fourth channel configured to accommodate a guidewire.

19. An endovascular probe, comprising:
an image recording device;
a bi-modal mechanism that in a first mode selectively provides irrigation and in second mode selectively provides suction; and
a laser fiber.

20. The endovascular probe in claim 19, further comprising an extendable inflation device.

21. The endovascular probe in claim 19, further comprising:
a probe body member surrounding the image recording device, bi-modal mechanism, and laser fiber; and
a balloon connected to the probe body member and configured to hold the probe body member in a fixed endovascular position when inflated.
